(19) United States
(12) Patent Application Publication
(10) Pub. No.: US 2015/0060311 A1
(43) Pub. Date: Mar. 5, 2015

(54) PACKAGE OF CONDOMS

(71) Applicant: Alexis Paolo Mendoza da Silva, Ciudad del Este (PY)

(72) Inventor: Alexis Paolo Mendoza da Silva, Ciudad del Este (PY)

(21) Appl. No.: 14/013,575

(22) Filed: Aug. 29, 2013

(51) Int. Cl. A61F 6/00

(52) U.S. Cl. CPC A61F 6/005
USPC 206/69

(57) ABSTRACT

The present invention refers to a package of condoms of any shape, size or composition characterized by the particularity of being provided with phosphorescent material or any other material having the same qualities and that relies on any type of relief of any material, shape, size, or composition, applied jointly or separately to a package of condoms, that allows to know the position in which the condom is inside the package.

### INVENTION FIELD

More particularly, the present Invention refers to improvements in packages of condoms for use in dark or low light conditions, and for visually impaired people; particularly, to facilitate the procedure of fitting of condoms on the erect penis at the moment of sexual relation, indicating the position of the condom inside the package before opening it, allowing to know the direction for correct and immediate fitting thereof.

### PRIOR ART

In the existent market, the models of packet of condoms are standardized based on predetermined features, in order to preserve the physical properties for their correct operation, avoiding contamination and maintaining integrity so that the product may meet all the requirements for such a purpose. Especially in regard to maximum efficiency in safety required for its use in sexual relations, assisting in the prevention of sexually transmitted infections and unplanned pregnancy.

However, they do not contain means for allowing to know beforehand and without opening the package, the direction or position in which the condom is, hindering the proper fitting, besides presenting a higher risk of contamination with pathogen agents. Therefore, the prior art lacks means to allow easier and safer fitting of condoms.

### OBJECTS OF THE INVENTION

The main purpose of the invention is allowing to identify the correct direction in which the condom is positioned before opening the package in the moment of its use, by means of drawings with fluorescent or phosphorescent materials and tactile dots in relief, applied outside the package, that allow their visualization or touch in all types of environmental conditions, mainly in dark or low light conditions, when the correct collocation of condoms becomes difficult, all of this in order to increase efficacy and safety of the condom to be used. It offers advantages in relation to the prior art, that does not foresee said difficulty.

The second object of the invention is allowing the safe use of condoms by visually impaired people, through tactile dots in relief, that will allow to identify the correct direction of the condom inside the package, making easier the fitting thereof.

The third object consists in indicating upon one or two fluorescent or phosphorescent lines, with tactile reliefs located on one or both sides of the condom's package, that allow opening the package in a safe manner, thereby reducing the risk of damage to the condom to be used.

The fourth object aims to optimize the fitting time of the condom on the erect penis at the moment of sexual relation, thanks to the fluorescent or phosphorescent drawings and dots in tactile reliefs that inform beforehand, by visual sense or tactile sense, the position of the condom inside the package, factors that, when opening and removing the condom, allow faster fitting thereof.

The fifth object is reducing the manipulation of the condom at the moment of use, enhancing asepsis and safety aiming at low risk of contamination by pathogen agents, which fact is due to incorrect manipulation; contributing to increase the prevention properties of the condom, namely, the prevention of sexually transmitted infections and unplanned pregnancy.

The sixth object consists in reducing the number of condoms discarded by incorrect fitting, due to the fact that manufacturers recommend throwing away the condoms that have been incorrectly fitted, due to the danger of contamination of those who are subject to contact with genitals.

The seventh object is providing for users greater comfort and confidence when using condoms in their sexual relations.

### DESCRIPTION OF DRAWINGS

For better understanding of the present invention, follows a detailed description thereof, in reference to accompanying drawings and pictures, wherein:

FIG. 1: represents the object of the present invention on the incorrect side of the package, which has no figure or relief, where the condoms in its interior are in an inadequate position to unroll to the base of the user's penis, if he wore the condom, i.e., facing the user;

FIG. 2: shows a perspective view which the user will have from fluorescent or phosphorescent drawings of the package of FIG. 1 that will allow him to visually notice the correct position of the condom inside the package and the dots in relief that will allow him to know the correct position of the condom by tactile sensing, allowing the user to know the direction in which he must unroll the condom that he will fit in himself.

FIG. 3: shows digital images of the object of the invention in a lighted environment, where the fluorescent or phosphorescent drawings and the tactile dots of relief may be appreciated, representing the correct position for the condom to be unrolled on user's penis.

FIG. 4: shows digital images of the object of the invention in unlighted environment exemplifying the material's phosphorescence with different drawings and a phosphorescent line that shows the user where to open the package of condom in a safe manner, preventing possible damages to the condom in the moment of opening the package.

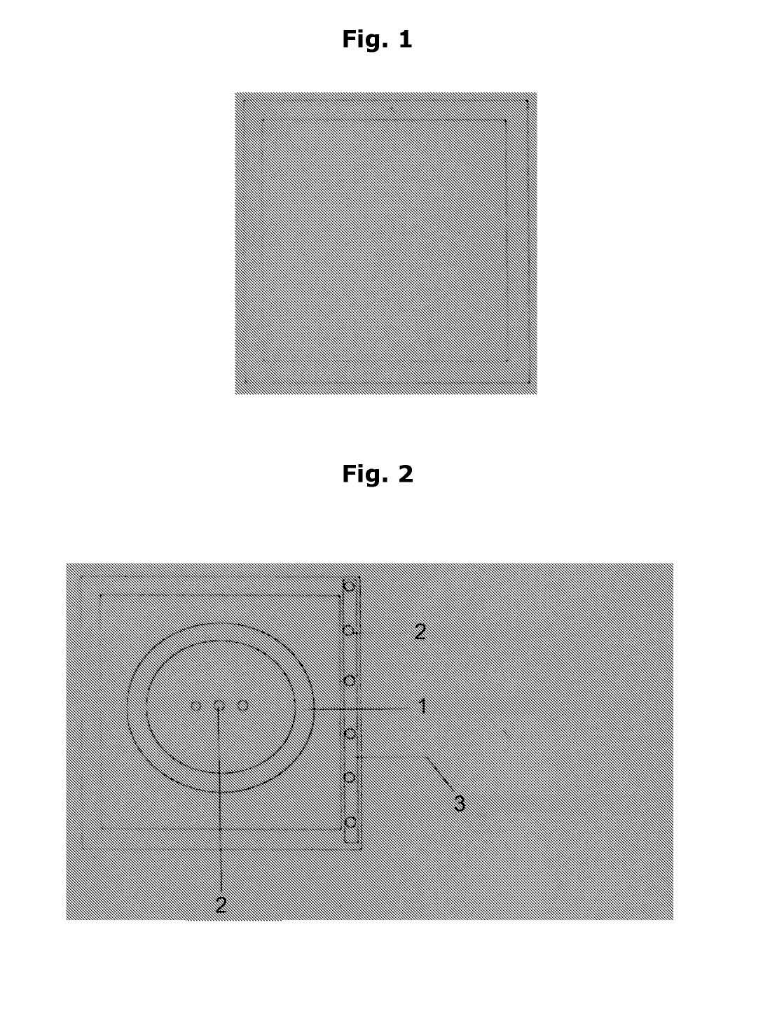

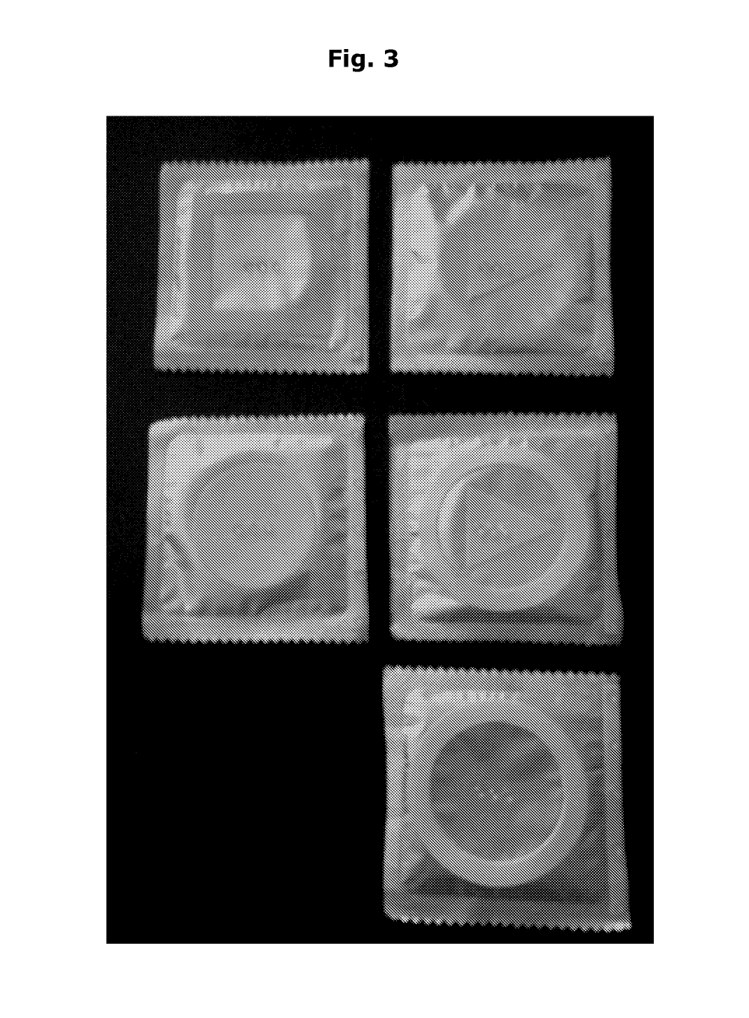

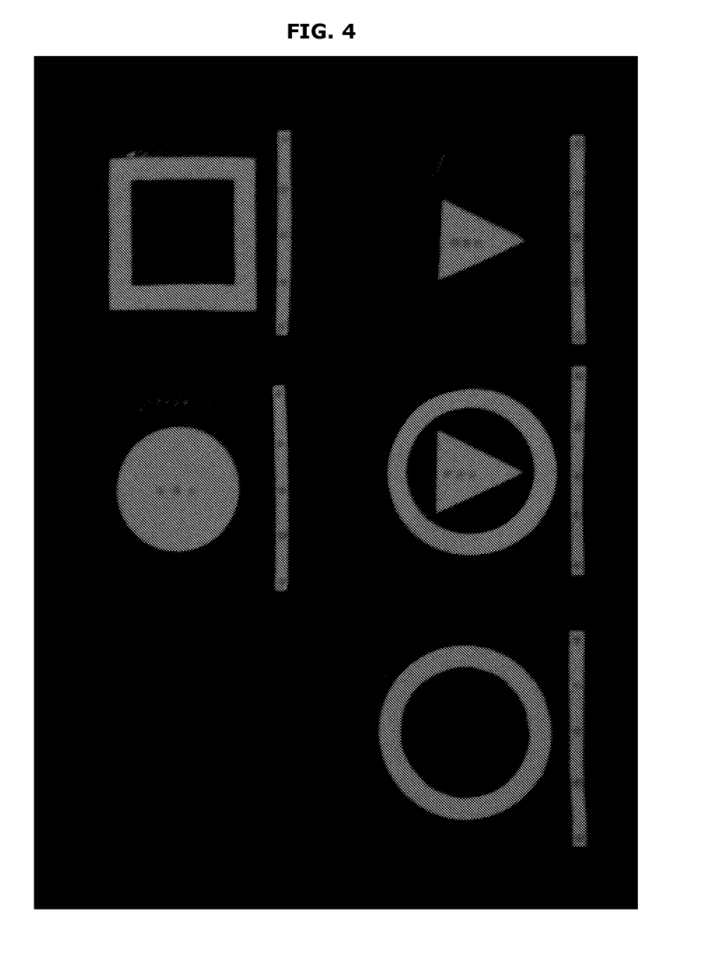

OBSERVATION: a detail that cannot be seen in the drawings and pictures is the transparent tape that covers the condom on the side wherein the drawings and fluorescent or phosphorescent dots are, which allows additional protection against possible damages thereto.

### DETAILED DESCRIPTION OF THE INVENTION

According to the illustrations and pictures and details thereof, the present invention, IMPROVEMENT IN PACKAGE OF CONDOMS, was particularly developed to facilitate the use or fitting of condoms by the drawings with fluorescent or phosphorescent material (1) and tactile dots in relief (2) that allow users to know by visual sense and by tactile sense the position in which the condom is inside the package so that the condom may be unrolled on users penis in a correct way.

The drawings made of fluorescent or phosphorescent material (1) allow to notice the position in which the condom is inside the package by visual sense through the light irradiation which said materials emit, by physical reaction to any light source, since they may absorb and store lighting energy when exposed to any conventional light source (daylight or electric light) and emit thereof in darkness during long periods of time.

In other words, they charge themselves in a few minutes and their effects last for hours. Their process of charge-discharge is completely reversible, by which they may recharge themselves as many times as desired. They emit light without any type of human, mechanical or electrical intervention. It is precisely this quality that renders them useful for the destination of this invention.

The tactile dots in relief (2) allow to notice the position in which the condom is inside the package by tactile sense; these consist of small plastic, silicon dots or of other materials with similar characteristics, that adhere themselves to the package and to the drawings resulting in tactile reliefs which complement the function of fluorescent or phosphorescent drawings; in the case wherein the visibility thereof has passed, the reliefs allow to know the correct position by tactile sense, considerably increasing utility and safety thereof, including permitting the use thereof by visually impaired people.

The invention further comprises one or two fluorescent or phosphorescent lines on one or both sides of the condom, with tactile dots of relief (3) that indicate adequate places at the package of condom for correct opening thereof, reducing possible damages to physical integrity of the condom at the time of opening.

Observation: a detail that is not noticeable in the pictures, by its transparency, consists of an adhesive film that covers the drawings, lines and reliefs of the package of the condoms where these are fixed on, increasing protection thereof against damages that they may suffer by manipulation or by the course of time and not focusing on the role for which they comply.

Observation: the sizes of the drawings and lines and of the tactile dots in relief may vary depending on the size of the package of condoms.

1. Package of condoms, applied to every cover of the condom or condoms, characterized in that they include fluorescent or phosphorescent tactile dots of relief, adhered to package of condoms, jointly or separately, that allow the user to know by visual sense or tactile sense, the position in which the condoms are located inside the packet.

2. Package of condoms, according to claim 1, characterized in that they contain all types of designs, of any size, shape or composition to be applied to packages of condoms, made of fluorescent or phosphorescent materials or any other that have the same qualities thereof.

3. Package of condoms, according to claim 1, characterized in that they contain all types of relief, of any material, shape, size or composition, that may be applied to packages of condoms and which may be sensed by tact.

4. Package of condoms, according to claim 2, characterized in that they contain all types of relief, of any material, shape, size or composition, that may be applied to packages of condoms and which may be sensed by tact.
